(19) United States
(12) Patent Application Publication
(10) Pub. No.: US 2022/0151515 A1
(43) Pub. Date: May 19, 2022

(54) FIBRE BASED SENSOR INCORPORATING ELECTROCHEMICAL SENSING

(71) Applicant: Imperial College Innovations Ltd, London (GB)

(72) Inventors: Guang-Zhong Yang, London (GB); Salzitsa Anastasova-Ivanova, London (GB); Burak Temelkuran, London (GB); Mohamed E.M.K. Abdelaziz, London (GB); Fabien Sorin, London (CH)

(21) Appl. No.: 17/442,260

(22) PCT Filed: Mar. 31, 2020
(86) PCT No.: PCT/GB2020/050859

§ 371 Date: Sep. 23, 2021

(30) Foreign Application Priority Data
Apr. 1, 2019 (GB) 1904554.1

(51) Int. Cl. A61B 5/1455; G01N 27/30; G01N 33/543; G01N 27/327

(52) U.S. Cl. CPC A61B 5/1455; G01N 27/3275; G01N 33/5438; G01N 27/305

(57) ABSTRACT

A sensor comprising an elongate member comprising an electrochemical sensor comprising an electrochemical filament extending along the length of the elongate member, wherein the elongate member comprises a fibre formed from a drawable material.

This invention relates to a sensor, and particularly, but not exclusively to a fibre based sensor incorporating electrochemical sensing. The invention also relates to a fibre based electrochemical sensor incorporating optical sensing.

A fibre based sensor having electrochemical or both electrochemical and optical sensors incorporated within the fibre may find particular application within the medical arena although other applications are also envisaged. Such a sensor may be used for diagnostic purposes.

It is known to introduce conducting elements into polymer fibres. However in such known sensors, it is not possible to take measurements to detect both electrochemical and optical factors.

According to a first aspect of the present invention there is provided a sensor comprising an elongate member comprising an electrochemical sensor comprising an electrochemical filament extending along the length of the elongate member, wherein the elongate member comprises a fibre and the fibre is formed from a drawable material.

The electrochemical filament may extend entirely along the length of the elongate member, or only partially.

By means of the present invention it is possible to have an electrochemical sensor at a tip of the elongate member. Such an arrangement is beneficial for the detection of precise and tiny concentrations of particles in parts of the body such as the bronchia, the gut and the small intestine.

In embodiments of the invention, the sensor further comprises an optical sensor comprising an optical filament extending along the length of the elongate member.

Such embodiments of the invention which are capable of sensing both electrochemical and optical variables in one sensor benefit from huge advantages in the detection of analytes that are optically detectable in addition to electrochemical analytes.

The optical filament may extend entirely along the length of the elongate member, or only partially.

The drawable material from which the elongate member is formed may comprise, for example a drawable polymer material. A wide range of suitable materials exists, and in embodiments of the invention, the fibre is drawn from a drawable amorphous thermoplastics material such as Polystyrene (PS), poly methyl methacrylate (PMMA), Acrylonitrile butadiene styrene (ABS), Polycarbonate (PC), Cyclic olefin copolymer (COC), Polycarbonate Alloys (PC/ABS, PC/PMMA), Polysulfone (PSU), Polyphenylsulfone (PPSU), Polyetherimide (PEI).

An advantage of having a sensor comprising a fibre formed from a drawable material is that the length and dimensions of the sensor may be readily tailored to achieve a sensor having appropriate dimensions.

The electrochemical sensor may be formed from any suitable material, and in embodiments of the invention, the electrochemical sensor is formed from an electrically conductive filament.

The electrically conductive filament may be an amorphous metal or polymer or may be a crystalline metal or polymer.

In an embodiment of the invention, examples of suitable materials for forming the electrochemical sensor include carbon.

In other embodiments of the invention the electrochemical sensor comprises an amorphous or crystalline metal or polymer which is made conductive by loading nano-particles such as carbon or nano-tubes such as carbon MT or Pt MT, or a combination of these materials.

Other suitable suitable metals include Pt, Ir, Gold, alloys of these materials and other similar materials and alloys.

The optical sensor may be formed from any suitable material, and in embodiments of the invention the optical sensor is formed from an optically transparent filament.

In embodiments of the invention, the optical sensor is formed from optically transparent polymers such as a Polycarbonate (PC), Cyclic Olefin Copolymer (COC), Poly methyl methacrylate (PMMA).

A suitable polymer must be not only be optically transparent, but also adapted to be co-drawn with another material such as silicone in order to form silica fibres containing the optically transparent polymers.

In embodiments of the invention the optical sensor is formed from polymers that are optically transparent at predetermined wavelengths. The predetermined wavelengths will vary depending on the specific analyte to be detected, and the dye being used to detect the analyte.

Alternatively, the optical sensor may be formed from a silica fibre.

In embodiments of the invention the sensor comprises a plurality of electrochemical sensors and a plurality of optical sensors.

In such embodiments of the invention, each of the electrochemical sensors may be formed from an electrochemical filament comprising one or more of the materials described hereinabove with reference to the electrochemical sensor, and each of the optical sensors may be formed from an optical filament comprising one or more of the materials described hereinabove with reference to the optical sensor.

In embodiments of the invention, each electrochemical and optical filament comprises at least one exposed area. In other words, each of the filaments comprises an area that is not enclosed within the elongate member.

The exposed area may be positioned at any convenient part of the elongate member and may for example be at an end of the elongate member, or at a side portion of the elongate member. The position of the exposed area will be determined by the application to which the sensor is to be put.

The sensors may be positioned in any convenient location such as at the tip of the elongate member, at various positions on a side of the elongate member and/or inside within a recess of the fibre.

Having sensors in a plurality of positions will result in a huge sensing area which will enable tests and detections to be carried out over a larger area than would be the case were the sensor to be positioned in one location only.

A further advantage of positioning sensors in a plurality of different positions along the fibre such as for example inside the fibre is that the sensing membrane is protected and biofouling is avoided. The life of the sensor is thus prolonged.

In embodiments of the invention, the electrochemical sensor comprises a working electrode. Such an electrode may be used to take electrochemical measurements.

In embodiments of the invention, the elongate member further comprises a reference sensor, which reference sensor comprises a reference electrode.

Measurements may then be taken using both the working electrode and the reference electrode.

In embodiments of the invention, the elongate member further comprises an auxiliary sensor, which auxiliary sensor comprises an auxiliary electrode.

In such embodiments of the invention, the sensors may be used together to take appropriate measurements.

In embodiments of the invention, the sensor may comprise a plurality of electrochemical and optionally optical filaments extending through the elongate member, the elongate member having one or more exposed areas at a distal end, and/or on a side of the elongate member, and/or inside the elongate member, which one or more exposed areas is functionalised to allow electrochemical and optionally optical detection of target molecules.

By means of such embodiments of the invention, it is possible to provide a sensor in which an electrical connection may be provided through a single fibre forming the elongate member. This is because all necessary electrodes may be formed within the elongate member which is in the form of a fibre.

The one or more electrochemical sensors may be prepared through electrochemical deposition of a sensing cocktail. The sensing cocktail may be any appropriate solution, and may for example be a solution appropriate for sensing glucose, lactate, pyruvate, hydrogen peroxide, dopamine, pH, sodium or potassium.

The one or more optical sensors may be prepared using a precise needle drop casting method in which a sensing membrane incorporating an appropriate dye is immobilised.

According to a second aspect of the present invention there is provided a method of forming a sensor comprising a electrochemical sensor wherein the sensors comprises a filament extending along the length of the elongate member the method comprising the steps of:
a. selecting a material to form a preform;
b. incorporating electrochemical sensor material into the preform; and
c. drawing the preform to form the elongate member and the electrochemical sensor.

The material selected to form the preform may be any convenient material, and in embodiments of the invention, the material comprises a drawable amorphous thermoplastics material.

In embodiments of the invention, the material selected is chosen from Polystyrene (PS), Poly methyl methacrylate (PMMA), Acrylonitrile butadiene styrene (ABS), Polycarbonate (PC), Cyclic olefin copolymer (COC), Polycarbonate Alloys (PC/ABS, PC/PMMA), Polysulfone (PSU), Polyphenylsulfone (PPSU), Polyetherimide (PEI).

In embodiments of the invention, the method comprises the further step, after the step of selecting a material to form a preform, of incorporating conductive metals and optionally optical sensor material into the preform.

The step of incorporating an electrochemical sensor material into the preform may take place before, after, or at the same time as the step of incorporating optical sensor material into the preform.

The electrochemical sensor material and the optical sensor material may be incorporated into the preform by any convenient method. Suitable methods include:
co-feeding the appropriate material into the preform during the drawing process;
co-drawing the appropriate material with the preform.

The preform may be formed by any convenient method. Examples of such methods include: hot press, cast moulding or injection moulding of thermoplastic pellets in a vacuum; the use of additive manufacturing techniques (3D printing); direct machining of commercially acquired rods or bars; and/or rolling of thermoplastics sheets/films and consolidating into preforms.

The preform may be formed by one or a combination of methods of the type listed above.

Once the preform has been fabricated and appropriate materials have been incorporated into the preform, the drawing process may take place.

In embodiments of the invention, the preform is a microscopic preform having dimensions of between 5 and 100 mm in diameter.

In embodiments of the invention, the cross-section of the preform remains substantially unchanged throughout the drawing process. This means that the resulting sensor has a cross-section which is substantially the same as the cross-section of the preform before the drawing process began.

The invention will now be further described by way of example only with reference to the accompanying drawings in which:

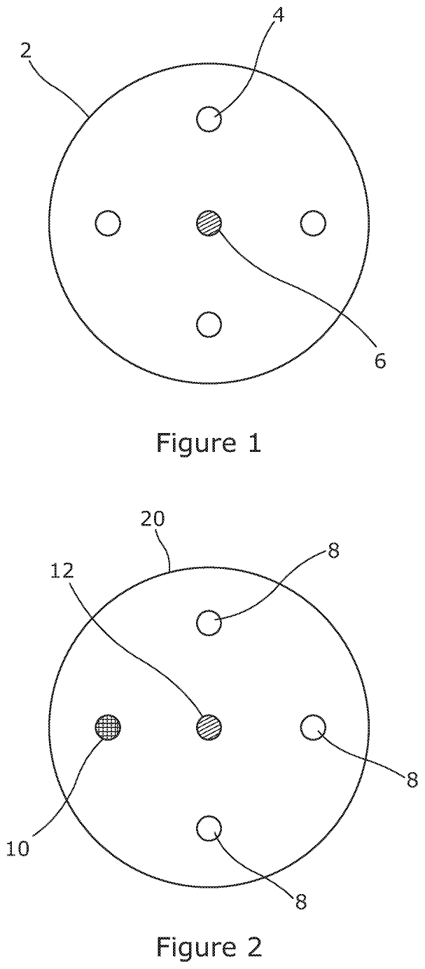

FIG. 1 is a schematic representation showing a first embodiment of the invention for ion sensing;

FIG. 2 is a schematic representation of a second embodiment of the invention suitable for enzymatic sensing;

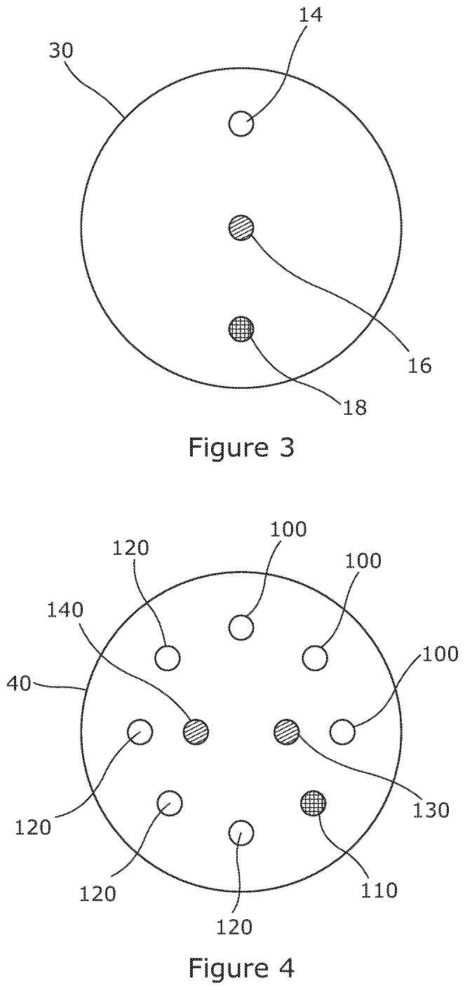

FIG. 3 is a schematic representation of a third embodiment of the invention for affinity-based sensing;

FIG. 4 is a schematic representation of a fourth embodiment of the invention suitable for both ion sensing and enzymatic sensing;

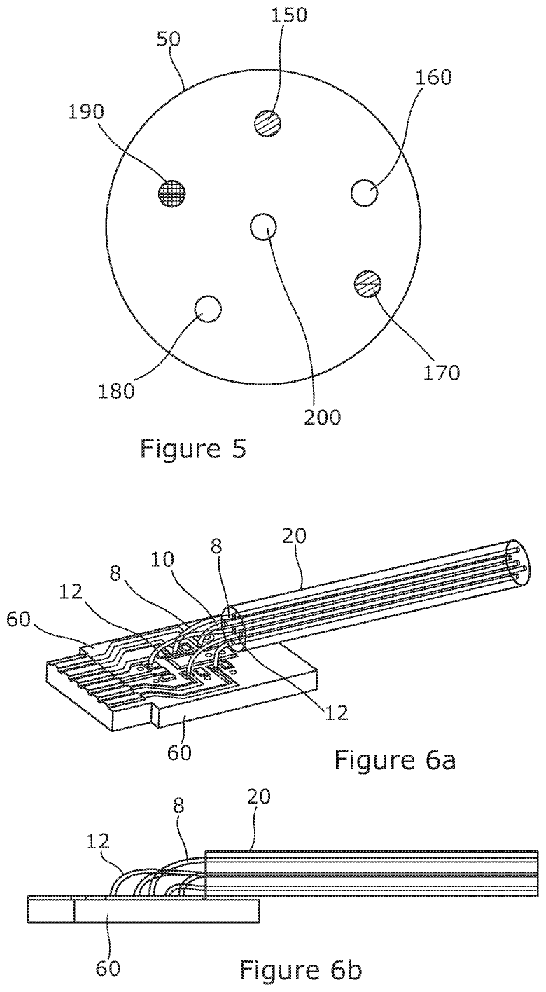

FIG. 5 is a schematic representation of a fifth embodiment of the invention comprising a sensor adapted to sense ions, enzymatic and affinity-based biosensing;

FIGS. 6a and 6b are schematic representations of a further embodiment of the invention showing a PCB interface connecting the sensor to a control module;

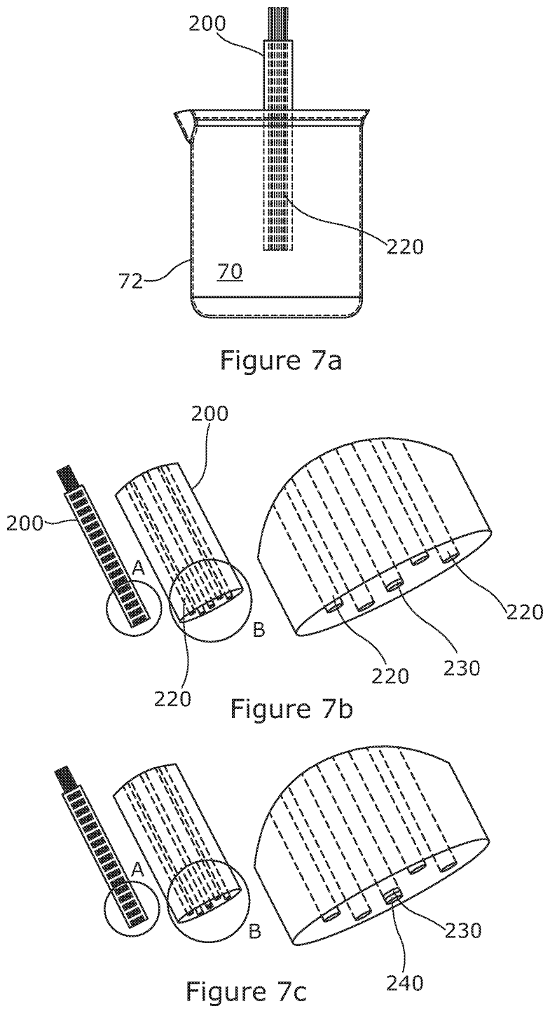

FIGS. 7a to 7c are schematic representations showing the process of selective fibre functionalisation in embodiments of the invention;

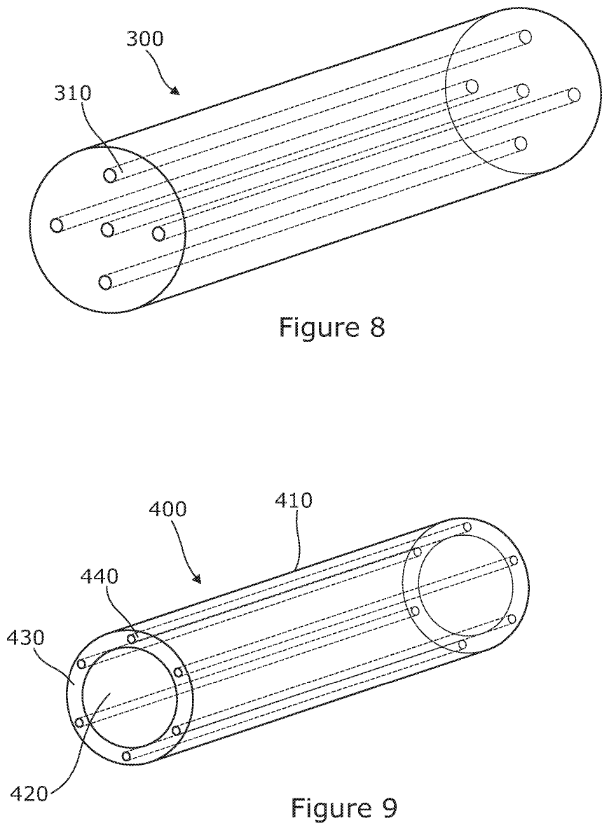

FIG. 8 is a schematic representation showing another embodiment of the invention in which a sensor is exposed at one end of the sensor;

FIG. 9 is a schematic representation of yet another embodiment of the invention in which the sensor comprises a catheter;

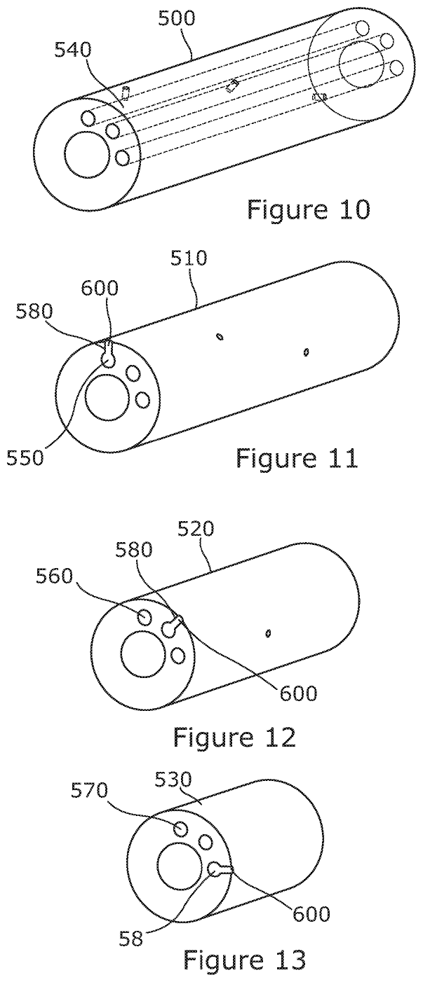

FIGS. 10 to 13 are schematic representations showing how sensors may be placed at different positions within a fibre in embodiments of the invention;

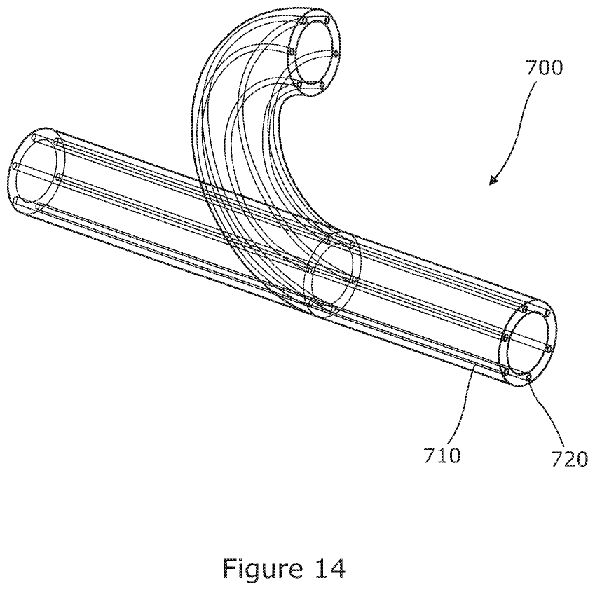

FIG. 14 is a schematic representation of a further embodiment of the invention comprising a steerable catheter integrated with sensing.

Referring to FIG. 1 a sensor according to a first embodiment of the invention is designated generally by the reference numeral 2. The sensor 2 is in the form of a single fibre formed from a drawable material. The drawable material may comprise a drawable polymer material. Suitable materials include amorphous thermoplastics material such as Polystyrene (PS), Poly methyl methacrylate (PMMA), Acrylonitrile butadiene styrene (ABS), Polycarbonate (PC), Cyclic olefin copolymer (COC), Polycarbonate Alloys (PC/ABS, PC/PMMA), Polysulfone (PSU), Polyphenylsulfone (PPSU), Polyetherimide (PEI).

The sensor 2 comprises a plurality of filaments 4, and in this embodiment the sensor comprises four electrochemical filaments 4, and a reference filament 6. The electrochemical filaments 4 each comprise ions working electrodes and may be formed from a platinum-iridium alloy. The reference filament 6 is an ions reference electrode and is formed from stainless steel.

Turning now to FIG. 2, a sensor according to a second embodiment of the invention is designated generally by the reference numeral 20. In this embodiment the sensor is again formed from a fibre which itself is formed from a drawable material. Suitable materials are the same as those described above with reference to the FIG. 1 embodiment.

Sensor 20 is an amperometric sensor and is therefore adapted to sense and measure metabolites such as lactate, glucose, pyruvate. The sensor 20 comprises three electrochemical filaments 8 which each comprise an enzyme working electrode formed from platinum. The sensor further comprises a filament 10 which is an enzyme counter electrode or auxiliary electrode which is also formed from platinum. Finally, the sensor 20 comprises a filament 12 which is an electrochemical reference electrode formed from stainless steel.

Turning now to FIG. 3, a sensor according to a third embodiment of the invention is designated generally by the reference numeral 30. This sensor is also formed from a fibre formed from a drawable material of the type described hereinabove with reference to FIGS. 1 and 2. The sensor 30 is affinity based sensor. An affinity based sensor is an analytical device composed of a biological recognition element such as an antibody, receptor protein, biomimetic material, or DNA interfaced to a signal transducer which is proportionate to the analyte concentration. The sensor 30 further comprises an electrochemical filament 14 which is a biomarker sensor based on antigen/antibody (APTAMER) detection and is a working electrode. The electrode is formed from carbon nanotubes loaded with polycarbonate. The sensor 30 further comprises filament 16 which is in the form of a counter electrode or auxiliary electrode formed from platinum and a filament 18 which is a reference electrode formed from stainless steel.

A sensor 40 according to a fourth embodiment of the invention is illustrated schematically in FIG. 4. The sensor 40 is a fibre formed from a drawable material as described hereinabove with reference to FIGS. 1 to 3. The sensor 40 is adapted to measure and analyze ions and enzymes. The sensor 40 comprises three fibres 100 which each comprise an enzyme working electrode formed from platinum, and a filament 110 which is an enzyme counter electrode, or auxiliary electrode also made from platinum. The sensor 40 further comprises four filaments 120 which each comprise an ion working electrode formed from a platinum-iridium alloy, and an ion reference electrode 130 formed from stainless steel. Finally, the sensor 40 comprises an enzyme reference electrode 140 formed from stainless steel.

Referring now to FIG. 5, a fifth embodiment of the invention is shown comprising a sensor 50. The sensor 50 is formed from a drawn fibre of the type described hereinabove with reference to FIGS. 1 to 4. The sensor 50 is adapted to measure and analyze ions and enzymes and is adapted to carry out affinity based biosensing within a single fibre. The sensor 50 comprises an ion reference electrode 150 formed from stainless steel, and ion working electrode 160 formed from a platinum-iridium alloy, an enzyme reference electrode 170 formed from stainless steel, an enzyme working electrode 180 formed from platinum and an enzyme counter electrode or auxiliary electrode 190 also formed from platinum. The sensor 50 further comprises a biomarker sensor 200 made from carbon nanotubes loaded with polycarbonate. This biomarker sensor 200 is based on antigen/antibody (APTAM ER) detection working electrode.

The enzyme working electrode 180 and the enzyme counter electrode 190 may also be used to sensor biomarkers and/or bacteria.

Turning now to FIGS. 6a and 6b, a fibre PCB interface is shown schematically. In this embodiment, the sensor 20 is of the type shown in FIG. 2 and described hereinabove, but a sensor according to any embodiment of the invention may be connected using a PCB interface as shown in FIGS. 6a and 6b.

As shown in FIGS. 6a and 6b, the sensor 20 may be connected to a PCB board 60 in order to electrically connected to the sensor 20 to for example an analyzer (not shown).

The ends of each of the filaments 8, 10, 12 may be soldered to appropriate parts of the PCB board 60 in order to achieve appropriate electrical connections.

Turning now to FIGS. 7a to 7c, the process of selective fibre functionalization is illustrated schematically.

As shown in the figures, a sensor according to embodiments of the invention comprises a sensor 200 according to embodiments of the invention, which sensor 200 comprises a fibre formed from a drawable material as described hereinabove with reference to the previous embodiments. The sensor 200 comprises filaments 220 which function as sensors as will be described below. The sensor 200 is inserted into a solution 70 in a container 72. The solution 70 is formed from predetermined compounds having predetermined concentrations so that the sensor may be appropriately calibrated.

FIG. 7b shows in more detail the filaments 220, forming part of the probe 20. Each of the sensors 220 is prepared according to the use to which the probe is to be put.

For an ions selective sensor, the sensors are initially cleaned and dried before a material such as platinum 230 is applied using for example nanoparticle deposition. Such a process results in an increased surface area of the sensors which may be linked to higher sensitivity of the sensors.

FIG. 7c shows how the fibre may be functionalized with several different layers 230, 240 on each of the sensing tips.

A further layer may be deposited, which layer contains a sensing membrane. The ions sensing membrane contains ionic sites such as nitrophenyl octyl ether, ionophores specific to the ion of interest such as pH, sodium, potassium, calcium, lead, iron, magnesium ionophores; placticizers such as polyvinyl chloride; solvent such as tetrahydrofuran.

Such a mixture (or cocktail), may be deposited on the sensors and left to dry overnight.

Following this step, the membrane is conditioned or charged. During such a process, a low and high concentration of the analyte to be tested are exposed to the membranes so that the sensor may be sensitive within a required range of interest.

For a sensor adapted to sense metabolites, the membrane may be prepared from an enzyme which is sensitive to an analyte of interest, which enzyme may be cross linked to bovine serum albumin using glutaraldehyde.

Several layers of biocompatible membrane layers such as polyurethane may be deposited after these processes in order to protect the sensors and enable the sensors to have an appropriate response during the lifetime of the probe 200.

Turning now to FIG. 8, a schematic diagram of part of a sensor 300 according to another embodiment of the invention is illustrated. The sensor 300 comprises a plurality of filaments or wires 310 which could be electrochemical sensors or optical sensors or a mixture of the two. The sensors in this embodiment are exposed at one end of the sensor 300. The sensors shown in FIG. 8 may be functionalized as described above with reference to FIGS. 7a to 7c.

In FIG. 9 another embodiment of the invention is designated generally by the reference numeral 400. In this embodiment the sensor 400 is adapted for use as a catheter or drain. In this embodiment the sensor 400 is formed from a fibre 410 having a drain 420 which may be an extraventricle drain in the form of a central channel and extending axially through the fibre 410. The channel 420 is defined by a fibre wall 430 in the form of a band. The drain 420 may be used to deliver drugs. Formed in the fibre wall is a plurality of filaments 440 comprising sensors which in this embodiment are exposed at one end of the fibre 410. The sensors formed from the filaments 440 may be functionalized as described above with reference to FIGS. 7a to 7c in order to allow for precise continuous monitoring of, for example, an infection, sepsis or inflammation.

Turning now to FIGS. 10 to 13, further embodiments of the invention are illustrated. In each of the illustrated embodiments the sensor comprises a fibre 500, 510, 520 and 530 respectively. In each embodiment of the invention, the sensor is formed from an elongate fibre comprising a plurality of filaments 440, 450, 460 and 470 respectively. In each of the embodiments the filaments are exposed at one end of the respective fibre and are also positioned to one side only of the fibre. Each of the filaments 540, 540, 560 and 570 is exposed at one end of the respective fibre. In addition, in each of the embodiments illustrated in FIGS. 11, 12 and 13, there is filament 580 which is also exposed along a portion 600 of the curved surface of the fibre.

Turning now to FIG. 14, another embodiment of the invention is shown. In this embodiment, the sensor comprises a catheter 700 formed from an elongate member in the form of a drawn fibre 710 having sensors in the form of filaments 720 formed therein. The sensors provide sensing parts which are integrated along the fibre 710 and may be integrated with a steerable catheter.

1. A sensor comprising an elongate member comprising an electrochemical sensor comprising an electrochemical filament extending along the length of the elongate member, wherein the elongate member comprises a fibre formed from a drawable material.

2. A sensor according to claim 1 further comprising an optical sensor comprising an optical filament extending along the length of the elongate member.

3. A sensor according to claim 1 wherein the drawable material comprises drawable amorphous thermoplastics material such as Polystyrene (PS), Poly methyl methacrylate (PMMA), Acrylonitrile butadiene styrene (ABS), Polycarbonate (PC), Cyclic olefin copolymer (COC), Polycarbonate Alloys (PC/ABS, PC/PMMA), Polysulfone (PSU), Polyphenylsulfone (PPSU), Polyetherimide (PEI).

4. A sensor according to claim 1 wherein the electrochemical sensor is formed from an electrically conducting filament.

5. A sensor according to claim 2 wherein the optical sensor is formed from an optically transparent filament.

6. A sensor according to claim 1 wherein the electrochemical filament comprises at least one exposed area.

7. A sensor according to claim 2 wherein the optical filament comprises at least one exposed area.

8. A sensor according to claim 1 wherein the electrochemical sensor comprises a working electrode.

9. A sensor according to claim 1 wherein the elongate member further comprises a reference sensor, which reference sensor comprises a reference electrode.

10. A sensor according to claim 1 wherein the elongate member further comprises an auxiliary sensor, which auxiliary sensor comprises an auxiliary electrode.

11. A sensor according to claim 1 comprising a plurality of electrochemical and optionally optical filaments extending through the elongate member, the elongate member having one or more exposed areas at a distal end, and/or on a side of the elongate member, and/or inside the elongate member, which one or more exposed areas is functionalized to allow electrochemical and optionally optical detection of target molecules.

12. A method of forming a sensor comprising a electrochemical sensor wherein the sensors comprises a filament extending along the length of the elongate member the method comprising the steps of:
a. selecting a material to form a preform;
b. incorporating electrochemical sensor material into the preform; and
c. drawing the preform to form the elongate member and the electrochemical sensor.

13. A method according to claim 12 wherein the material selected to form the preform comprises a drawable amorphous thermoplastics material.

14. A method according to claim 12 wherein the material selected is chosen from Polystyrene (PS), Poly methyl methacrylate (PMMA), Acrylonitrile butadiene styrene (ABS), Polycarbonate (PC), Cyclic olefin copolymer (COC), Polycarbonate Alloys (PC/ABS, PC/PMMA), Polysulfone (PSU), Polyphenylsulfone (PPSU), Polyetherimide (PEI).

15. A method according to claim 12 further comprising the step of incorporating an optical sensor material into the preform.

16. A method according to claim 12 comprising the further step of exposing a surface of the or each sensor.

17. A method according to claim 12, wherein the preform has a diameter of between 5 and 100 mm.
